(19) United States
(12) Patent Application Publication
(10) Pub. No.: US 2002/0061842 A1
(43) Pub. Date: May 23, 2002

(54) Method for sterilizing a native collagen in liquid medium, sterile native collagen obtained, compositions containing it and uses

(75) Inventor: Hamza Mansour, Rosny-Sous-Bois (FR)

Correspondence Address: JACOBSON HOLMAN PLLC, 400 SEVENTH STREET N.W., SUITE 600, WASHINGTON, DC, 20004, US

(73) Assignee: OCTAPHARMA AG

(21) Appl. No.: 09/917,233

(22) Filed: Jul. 30, 2001

Related U.S. Application Data
(63) Continuation of application No. 09/646,893, filed on Oct. 6, 2000, now abandoned.
Application No. 09/646,893 is a 371 of international application No. PCT/FR99/00814, filed on Apr. 9, 1999.

(30) Foreign Application Priority Data
Apr. 10, 1998 (FR) 98/04531

(51) Int. Cl. A61K 38/39; A61K 38/14; A61K 31/496; A61K 31/47

(52) U.S. Cl. 514/8; 514/21; 514/253.08; 514/312

(57) ABSTRACT

The invention concerns a method for preparing native collagen, comprising pretreatment of the collagen in a mixer with double transverse cutters equipped with a system controlling agitating and shearing velocity and a thermostat, and a subsequent step of sterilizing the collagen in liquid medium. The invention also concerns a sterile collagen, in particular a collagen mostly of type I, in native state and in particular pharmaceutical and/or parapharmaceutical and/or medico-surgical and/or ophthalmologic and/or cosmetic compositions and applications thereof.

The present invention relates to a method for preparing collagen comprising a step of sterilization of said collagen, to the sterile collagen thus obtained, and to the compositions and uses, in particular the pharmaceutical and/or parapharmaceutical and/or medico-surgical and/or ophthalmological and/or cosmetic compositions and uses, of the latter.

Collagen and its uses have been generally described in reference works such as Collagen, Vol. 1, 2, 3 Marcel E. Nimni., CRC Press Inc, 1988 and Methods in Enzymology, Vol. 144, 1987 and Vol. 82, 1982, Ed. Leon W. Gunningham, for example.

It is well known that collagen is a molecule with a 3-dimensional structure which is present in most human and animal tissues, and is found, in particular, in the skin, the tendons and the placenta.

Its various known properties are, inter alia, moisturization, blood clotting and wound healing.

In order to preserve its hemostatic action, it is generally desirable for collagen to conserve its native form before its administration into the human body, as recalled, for example, in document U.S. Pat. No. 4,515,637.

Moreover, because of its uses, in particular its medical and/or cosmetic uses, the collagen must be free of any contamination (virus, bacterium, prion, etc.). The sterilization of collagen has been diversely examined in the prior art, which has been confronted, for a few years, with new challenges (risks of transmission of AIDS, Creutzfeldt-Jakob disease, etc., due to the natural origin of collagen.

Techniques for sterilizing collagen in solid form comprise the use of ethylene oxide according, in particular, to document FR-A-2,393,581 or irradiation (use of beta or gamma ionizing radiation) as described for example in document EP-A-0,224,453. The latter, which describes cosmetic and pharmaceutical uses of collagen-based sponge type, recommends sterilization in dry form due to the impossibility, according to that document, of sterilizing soluble natural collagen incorporated into emulsions or solutions.

Document EP-A-0,664,132 describes an adhesive composition, for surgical use, based on non-crosslinked collagen modified by oxidative cleavage. One preferred embodiment of the composition described in that document consists in preparing a sterile powder of non-crosslinked type I collagen [lacuna] by oxidative cleavage, and in dissolving this powder in sterile ultrafiltered water by heating to approximately 60° C. with stirring. It is specified that, in view of the controlled heating which is carried out, the collagen loses its helical structure and is transformed into gelatin. Moreover, it is explained that the adhesive compositions representing the state of the prior art in that document, which are based on crosslinked collagen, are not easy to handle and pose application and mechanical strength problems.

Document EP-A-0,667,352 describes an alkaline treatment used on collagens in solution for the purpose of eliminating possible prions. In order to avoid all risk of residual contamination by said prions, that document teaches, more specifically, a method consisting in solubilizing tissue-extracted collagen either by enzymatic digestion or by alkali cleavage of the covalent bonds which interlink the collagen chains, and then in removing the tissue debris by filtration and in subjecting the collagen thus solubilized to an alkali treatment.

Document FR-A-2,586,703 teaches the preparation of transparent and physiological gels or of collagen solutions based on type IV collagen. It is, however, recalled that type I, II, or III collagens, since they are practically insoluble at neutral pH, only allow the production of opaque suspensions, and that it is impossible to use them for preparing transparent and physiological gels without resorting to a chemical modification of the collagen molecules, which are likely to make them antigenic and/or toxic.

Knowledge of this prior art and investigation by research work have enabled the present inventor to demonstrate the following difficulties and problems which still remain to date in the preparation of sterile native collagen, in particular of type I:

Handling

High viscosity of native collagen, which imposes technical constraints on carrying out filtrations, in particular in procedures of direct filtration of conventional native collagen in liquid medium.

Denaturation

Partial or total heat denaturation of native collagen during the various usual sterilizing treatments, which causes the loss of all or part of its structure and/or of its beneficial properties such as resorption, mechanical strength or moisturization, for example.

Sterilization by irradiation (ionizing radiation) or with ethylene oxide, which causes the deterioration of advantageous active principles to be combined in solution with the collagen, such as antibiotics, for example, and/or which causes a conventional gel based on native collagen to separate into two phases, making it unsuitable for commercial use.

Denaturation by sustained alkali treatment.

Toxicity and/or contamination (viruses, prions)

Presence of traces of residual ethylene oxide in the products sterilized by this means/presence of contaminants in the crude starting collagen.

Incompatibility

pH incompatibility between the advantageous active principles which are soluble at neutral pH, such as anti-infectious agents, for example, and the collagen which is soluble in acid medium.

Galenical Formulation

Unsatisfactory presentation of preparations based on native collagen, having a fibrous and heterogeneous appearance.

Presentation of conventional preparations of biological adhesive, which is unsuitable given the use for which they are intended (such as preparations which are not ready to use and which require too long a preparation time, for example).

Thus, the aim of the present invention is to overcome the abovementioned difficulties and to resolve the abovementioned problems, in particular the sterilization of collagen in liquid medium while at the same time preserving its native form, and other problems which will be set out hereafter.

The subject of the present invention is thus a method for preparing collagen in native form, comprising a step of pretreating collagen in a mixer with double transverse cutters which is equipped with a system for controlling the stirring speed and shear rate and with a thermostat, and a subsequent step of sterilizing collagen in liquid medium.

A subject of the present invention is also a sterile collagen, in particular a collagen mostly of type I, in native form and compositions for human and/or veterinary use which contain it as the only active principle, or in which it is combined with other substances advantageous which are acceptable from a pharmaceutical, parapharmaceutical, medico-surgical, and/or ophthalmological and/or cosmetic point of view.

A subject of the invention is also the use of collagen in accordance with the invention in the manufacture of a composition intended for pharmaceutical and/or parapharmaceutical and/or medico-surgical and/or ophthalmological and/or cosmetic treatment of a human or animal body.

These subjects and others will become apparent to persons skilled in the art upon reading the detailed description which follows.

The methods for preparing sterile collagen which are currently available pose the problems of implementation already mentioned above and, to date, have not, in practice, allowed a direct sterilization of collagen in liquid medium alone, or in the presence of other active principles, which is simple to carry out and which especially effectively guarantees the sterility and the native nature of the collagen obtained, thus preserving its beneficial properties.

Thus, the present invention has achieved this objective, and relates to a method for preparing collagen comprising a step, prior to the sterilization, of mechanical pretreatment, in a mixer, such as a “cutter mixer” sold under the brand DITO-SAMA 175 (LA BOVIDA), and modified by the company J.F.I. Graulhet, France, for example, which is equipped with a temperature control system, of an extract of nonsterile native collagen, which is solubilized and purified, optionally pepsinized. This pretreatment step allows, most surprisingly and unexpectedly, the sterilization in liquid form of the collagen and the preservation of its native form.

Preferably, the collagen thus prepared is a collagen mostly of type I.

The collagen mostly of type I thus prepared is sterile and in nonnative form, i.e. with unchanged secondary and tertiary structures, and having conserved all of its beneficial properties, even after sterilization.

The collagen in accordance with the present invention is useful in very many applications which are detailed later, in particular:

Human and veterinary medical applications:

pharmaceutical applications

parapharmaceutical applications

ophthalmological applications

medico-surgical applications

Cosmetic applications.

It can be used on its own, i.e. without any other active principle, for example in the form of a sterile clear solution,

for injectable preparations

or alternatively as a biological adhesive and in other forms which are detailed later.

It can be used in compositions in combination

at low pH, as a vehicle for other active principles for immediate release of the latter. These other active principles are advantageously chosen from the group consisting of clotting factors, for example thrombin, anti-infectious agents, for example metronidazole, antibiotics, in particular macrolides such as gentamicin, fluoroquinolones such as pefloxacin, anti-inflammatory agents, for example the acylcarboxylic or oxicam derivative steroidal or nonsteroidal anti-inflammatory agents, antimycotic agents and growth factors, in particular bone growth factors such as somatotropin and epidermal growth factors such as EGF.

However, in certain specific uses, for example in ophthalmology or in the case of medicinal combinations incorporating active principles which are incompatible with an acid pH, such as the glycopeptide antibiotic vancomycin, to cite just these two examples, it is obviously preferable to have a collagen at neutral pH.

It can also be advantageous to have collagen at neutral pH as a vehicle for a sustained release of active principles such as, for example, clotting factors, or alternatively, when a combination of the activity of the latter and a mechanical action is sought. Thus, it is also particularly advantageous to use the collagen in accordance with the present invention.

or by combining, in stratified solid forms, the two previous combinations at acid pH and at neutral pH in specific applications which will be described later.

By way of examples of the very many presentations of the collagen according to the invention, mention may be made of:

liquid forms: gel, in particular injectable gel, drop, foam, spray,

pseudosolid forms: films, sheets, membranes, bands or patches, etc.

solid forms: powders, laminated/stratified sponges, films, membranes, threads, sutures, etc.

It is also possible to exploit the acidity and solubility properties of the sterile collagen in accordance with the invention in pharmaceutical or cosmetic compositions, in particular in moisturizing compositions.

It is possible, for example, to use the collagen at low pH and in pseudosolid form obtained in accordance with the method of the invention, alone or combined with other active principles such as vitamin E or vitamin C, in particular as a tissue moisturizer on the epidermis and as a vehicle for active principle(s) in a face mask.

These presentations and some others will be illustrated in the remainder of the present description.

In the context of the present invention,

the term “native collagen” is intended to mean: a collagen which has undergone no modification in its initial structure, the integrity of its polypeptide chains being preserved (including the telopeptides) and the helical structure of the molecule having remained intact.

The term “atelopeptide collagen” is intended to mean: a collagen which has undergone limited proteolysis by enzymatic digestion using a proteolytic enzyme other than collagenase, such as, for example, trypsin, papain, etc., or even better still pepsin, leading to the removal of its telopeptides. Of course, if it is in native form, it conserves its trihelical structure.

The term “sterile collagen” is intended to mean: a collagen which is sterile in accordance with the standards of the World Health Organization, European Pharmacopea and US Pharmacopea (USP 22), i.e. which is certified sterile after microbiological analysis according to the criteria recognized by the administrations concerned.

The term “liquid collagen” is intended to mean: a collagen in solution or in the form of a suspension, a dispersion or a gel.

The term “pseudosolid collagen” is intended to mean: a collagen in the form of a gel which does not run and which forms a compact and homogeneous mass containing more than 75% water.

The term “solid collagen” is intended to mean: a collagen which contains no more than 10% to 25% water, and which can be in a solid, for example, powdered, film, sponge, membrane, etc., form.

The phrase “native collagen with polymerizing power” is intended to mean: a native collagen prepared according to a variant of the method in accordance with the present invention, which has, in liquid form or once solubilized from a solid form, a fluid nature at a temperature higher than 37° C. It transforms into a homogeneous mass and is in a “pseudosolid” form at a temperature less than or equal to approximately 37° C. Such a collagen can of course be atelopeptide in nature.

The phrase “solubilized and purified extract” is intended to mean: a nonsterile collagen extract at low pH (from 1.5 to 6.5, conventionally from 3 to 6, and even better still from 4 to 5), in which the sulfuric ash content is in general less than 2% and the lipid content is in general less than 1%, and which comprises from 12% to 13.9% hydroxyproline and from 17% to 18.7% total nitrogen, and which contains no trace of tryptophan or of polypeptide chains of less than 95,000 Da.

Such an extract can be obtained from dried limed fresh hides, such as calf or rabbit hides, and/or from connective tissues in particular from cattle, goats, pigs, sheep, horses, rabbits, stags, ostriches, fish, etc., or from dried or fresh animal bones, corneas or even tendons (for example ostrich Achilles tendons), or alternatively from purified collagen fibers or powders. It can also be extracted from placenta of human origin.

The extract which is useful in the context of the present invention is prepared by a series of treatments comprising, in summary, steps of:

liming/hair-removing/washing/deliming/removing the epidermis and the subcutaneous tissues/cutting up/spin-drying/immersing in 1N NaOH for 12 h at 25° C. /washing to totally eliminate NaOH/grinding/cutting up into films/defatting in a bath of Triton X100 at 0.01%/optionally eliminating aminoglycans in a bath of K₂HPO₄/washing/spin-drying/washing with purified water/spin-drying/acidifying in 10⁻²M acetic acid, monochloroacetic acid or citric acid/optionally diluting the collagen extract.

The extract can be in the form of an aqueous solution, of a suspension, of a dispersion, of a gel or of a paste, etc.

The method of the present invention can be applied to various type I, II, III, IV, etc. collagens. However, hereafter, and purely by way of nonlimiting illustration, the terms “collagen extract” and “collagen” refer, unless otherwise indicated, to a collagen extract or collagen mostly of type I.

Thus, in the context of the present invention, the collagen extract consists mostly of type I collagen, i.e. containing more than 90% of type I collagen. The extract can also consist totally, of type I collagen, for example by continuing the elimination of type III collagen according to the known techniques of dialysis, centrifugation and differential precipitation with sodium chloride.

Type I collagen is a high molecular weight polymer of 285,000 daltons consisting of the association in trihelical of two identical polypeptide chains called α₁(I)₂and of a polypeptide chain called α₂(I)₁. It belongs to the family of fibrillar collagens (type I, II, III and V).

Collagen has a denaturation peak which depends on its origin, on the extraction method leading to it and on its crosslinked or non-crosslinked form. In non-crosslinked form, the denaturation peak is at 35 to 45° C.

The collagen mostly of type I has an α₂(I)₁/α₁(I)₂ ratio of 0.48 to 0.52, as measured by densitometry carried out on polyacrylamide gel after migration of the chains by electrophoresis.

The viral inactivation of this extract and of the pepsin optionally present can be carried out by chemical treatment by passing alternately through buffer solutions at pH higher than or equal to 13 and at pH lower than or equal to 2.5. Treatments which are in themselves known are generally carried out, such as soaking in a bath of calcium hydroxide at 4% combined with sodium sulfide at 3%, followed by a second bath of sodium metabisulfite at 0.5% combined with ammonium chloride at 2%.

The optional neutralization of the pepsin is carried out by sodium treatment which brings the pH back to 6.8 to 7.4, as set out, for example, in document FR 2,586,703.

Similarly, the elimination of possible agents which are responsible for bovine spongiform encephalopathies, and which are called “prions”, can also be carried out in a known way, as described in the directives of the WHO (WHO/COS/VHP/92.104) and of the European Community dated Dec. 11, 1991, for example. This elimination can thus consist of a treatment with a solution of 1N sodium hydroxide for 1 h to 48 h at 25° C. or alternatively of a treatment with sodium hypochlorite for at least 1 h at 25° C.

According to a first aspect, a subject of the present invention is thus more precisely a method for preparing a collagen from a solubilized and purified extract of nonsterile native, optionally atelopeptide, collagen, characterized in that it comprises the steps consisting in mixing and shearing said extract in a mixer with double transverse cutters for a period of 1 min. to 60 min., conventionally from 15 to 40 min. and preferably from 10 to 20 min., at a stirring speed of 100 rpm to 10,000 rpm, in general from 500 rpm to 7000 rpm and preferably from 1000 rpm to 5000 rpm, while at the same time controlling the temperature, and then in sterilizing said extract, as a result of which a sterile collagen in native form is obtained.

The collagen thus obtained can be in the form of a liquid, of a gel, of a pseudosolid or of a solid.

Advantageously, according to a first implementation variant, and when the extract is not usually pepsinized (hereafter, “variant 1” will refer to, for simplicity, the embodiment of the method of the invention in which the extract is not pepsinized, except where otherwise indicated), the temperature, which is always controlled, does not generally exceed 40 to 50° C., or even 60 to 80° C. in the presence of a crosslinking-agent (according to another embodiment which is an alternative to this variant and is described later in detail).

Preferably, according to the first variant of the method of the present invention, since the extract is not pepsinized, its temperature is gradually increased from room temperature until 40° C. to 50° C. is reached in said mixer, while at the same time raising, in stages, the initial stirring speed from 500 rpm so as to reach up to 5000 to 7000 rpm, and at each phase of viscosity increase, a dilution of the extract is carried out. The collagen is thus maintained at a chosen high viscosity, while at the same time decreasing its concentration. Advantageously, said high viscosity is 17,000 to 20,000 cps. For example, when said maximum temperature is 42 to 44° C., said extract is left to stand before said sterilization step.

It is observed, after sterilization, that the collagen thus prepared has conserved its native form and has acquired improved properties, in particular of elasticity and of mechanical strength. It has adhesive and polymerizing power.

Preferably, said extract in said mixer is brought from room temperature to a temperature of at least 40 to 50° C. in stages of 3° C. to 5° C., while simultaneously increasing the stirring speed, also in stages of 500 to 1000 rpm, so as to reach a speed of 1500 rpm to 7000 rpm, preferably 5000 rpm.

It is in fact important to raise the temperature in stages, since an abrupt increase in temperature temporarily denatures the collagen without sufficiently increasing its elastic properties. This temperature is brought to at least 40° C., since below this temperature the subsequent filtration is made difficult or even impossible.

It is also important to increase the stirring speed in stages, so as to better control the chosen high viscosity while at the same time decreasing the collagen concentration, as already mentioned.

The collagen concentration in the extract is from 0.001% to 15%, in general from 0.1 to 10%, and preferably from 3 to 6%.

As described above, the temperature in said mixer can, however, reach higher values which can be as high as 80° C. in the presence of crosslinking agents such as glutaraldehyde and azyl acids such as hydrazine, or any other crosslinking agent which is compatible with collagen. When they are present, these agents have a final concentration in the extract of 0.00075% to 0.1%, preferably 0.0075% to 0.01%, in the case of glutaraldehyde, and of 0.1% to 2%, preferably 0.5% to 1.5%, for hydrazine. The collagen thus “crosslinked” consequently has a higher setting temperature which can be around 40 to 50° C., and has an in vivo resorption time which is longer compared with non-crosslinked collagen, and which can be as long as 8 weeks to 12 weeks.

According to a particular embodiment of this variant of the method according to the invention, which consists in obtaining the sterile native collagen in the form of a viscous liquid, the following procedure is carried out:

A nonsterile native collagen, which is extracted from rabbit hides or from ostrich Achilles tendons, is prepared at a sufficiently high concentration of about 10 to 15%.

The extract of nonsterile native collagen is then subjected to stirring and to shear in the mixer with double transverse cutters already mentioned, at a moderate speed of 500 to 1000 rpm and at room temperature, of about 20° C.

The spin speed and shear rate are increased in stages, which has the effect of greatly increasing the viscosity of the extract. This viscosity can have values of 7000 cps to 20,000 cps. From 1500 cps, the extract has a virtually elastic appearance, which makes its stirring and its shear in the mixer extremely difficult.

At each phase of viscosity increase, a dilution of the extract is carried out by adding water for injection (WFI) or 10⁻² M citric acid, and the stirring speed and temperature in the mixer are increased in stages until a very high viscosity of about 20,000 cps is obtained.

This operation is repeated several times, preferably four to five times until a temperature of 35° C., a speed of 3000 to 5000 rpmute and a collagen concentration of about 3 to 6% are reached.

The product is kept at this temperature of 35° C. and at this speed of 3000 to 5000 rpm for a few min., preferably 3 to 5 min.

The temperature in the mixer is increased until 40 to 50° C. is reached, preferably 40 to 45° C., in the case of non-crosslinked collagen. At this temperature, liquefaction of the extract is most surprisingly observed, with a drop in viscosity to approximately 40 to 100 cps.

The collagen extract, prepared as has just been set out above, is immediately thoroughly filtered, at a controled operating temperature of 40 to 50° C., through membranes with a porosity of 0.45μ to 0.22μ.

It then undergoes sterilization, preferably by an absolute filtration through a membrane with a porosity of 0.22μ, such as a Millidisck membrane sold by the company Millipore, at the controled operating temperature of 40 to 50° C. The filtration time at the temperature of 40 to 50° C. is chosen so as not to exceed 1 h, better still 10 to 20 min. It is also possible to carry out the sterilization, also preferably, by adding peracetic acid, produced in particular by the company Air Liquide under the name Soproper, at the final concentration in the thoroughly filtered extract of 0.5% to 50%, and preferably from 5% to 15%, by weight with respect to the dry weight of the collagen. The neutralization of this sterilizing agent is carried out by adding sodium thiosulfate in a proportion of 2 to 20 g per 1 g of peracetic acid, and preferably of 4 g to 12 g/g of peracetic acid. The contact time required for the sterilization by peracetic acid is from 1 h to 24 h at room temperature, preferably from 2 h to 4 h.

According to another particular embodiment of this first variant of the method in accordance with the present invention, which consists in obtaining the native collagen in the form of a pseudosolid gel, since the collagen concentration is from 0.5% to 10%, it is also advantageous to sterilize it directly by irradiation at a dose of 2.5 Mrad (beta or gamma ionizing radiation). It is observed, surprisingly, that the pseudosolid gel remains compact and homogeneous and that, therefore, no separation into two phases occurs, unlike conventional gels, and it conserves its physico-chemical properties and its polymerizing power. This pseudosolid gel sterilized by this method can advantageously be liquefied so as to be combined with sterile active principles which are sensitive to ionizing radiation, such as clotting factors, antibiotics, antimycotic agents, etc., for the production of liquid or solid preparations, in particular the preparations cited by way of examples below: sponges, powders, films, membranes, patches, etc.

When this method of sterilization is used on solid preparations of collagen which is obtained according to this first variant of the method, and which is on its own or combined with active principles which are resistant to ionizing radiation and which are chosen from the therapeutic classes cited in the present document, no modifications of the physico-chemical properties or of the polymerizing and adhesive power of this form of collagen occur.

According to this variant 1, the method according to the invention can also comprise a neutralization step. The neutralization can be carried out before or after the sterilization step. This neutralization can be carried out with sodium hydroxide, for example, in order to adjust the pH to 6.8 to 8.2, and preferably to 7.0 to 7.4. It is noted, most surprisingly, that this neutralization of the collagen in viscous liquid form and at a temperature of 40 to 50° C. does not cause any precipitation of the collagen. The extract remains in clear or translucent viscous liquid form, and rapidly sets under the same conditions as the collagen at low pH. It is very advantageous, since the production of certain preparations such as a gel based on collagen, or a combination of collagen with vancomycin, has been prevented to date because of the precipitation of the collagen at neutral pH.

The analyses of the collagen molecule of the invention by the conventional techniques already mentioned confirm the integrity of the primary, secondary and tertiary structures of the collagen thus neutralized.

Characteristics of the collagen obtained according to variant 1:

It is noted, most surprisingly, that the collagen sterilized in accordance with the first variant of the method of the present invention conserved its native form.

At a temperature of 40 to 50° C., it is a viscous translucent liquid.

Its viscosity varies in general from 20 cps to 300 cps, and preferably from 30 to 200 cps, and even better still from 40 to 100 cps.

Its pH is in general from 1.5 to 6.5, preferably from 2 to 5 and even better still from 3 to 4.

Its concentration is in general from 0.001 to 15%, preferably from 0.1 to 10% and even better still from 3 to 6%.

It sets very rapidly at a temperature lower than or equal to 38° C. It takes a pseudosolid form which has notable adhesive properties of hemostatic adhesive. Its setting time at a temperature of approximately 38° C. is less than 1 min. at a concentration higher than or equal to 5%, between 1 min. and 2 min. at a concentration of 3% to 4%, and approximately 3 min. at a concentration lower than 2%.

The native sterile collagen obtained also has the other important characteristics or properties which follow:

collagen mostly, i.e. containing 90% or more, of type I, i.e. an α₂(I)₁/α₁(I)₂ ratio which is from 0.48 to 0.52

water content: greater than 80%

appearance: liquid at a temperature higher than 38° C. approximately and sticky to the touch; pseudosolid compact and homogeneous gelled mass at a temperature lower than 38° C. approximately.

total nitrogen: 17.0% to 18.7%

hydroxyproline: from 12% to 13.9%

free of tryptophan, aminoglycans and polypeptides of molecular weight<95,000 daltons

lipids<1%

sulfuric ash<2%

denaturation peak: 38° C. to 45° C., as measured by differential thermography (DSC).

adhesive power

polymerizing power

conservation at room temperature

immediately ready to use.

Thus, a subject of the present invention is also such a collagen which has the abovementioned characteristics.

Preparations based on collagen obtained according to this first variant of the method in accordance with the present invention and uses:

As already mentioned, the collagen in accordance with the invention at acid or neutral pH has a polymerizing power and notable adhesive properties. The latter, combined with its hemostatic and resorption properties, confer upon it the quality of a resorbable hemostatic adhesive.

The collagen of the invention can be distributed preferably into syringes or vials of 2 to 5 ml which are preferably made of glass and which are kept at a temperature allowing it to polymerize or set of between 0° C. and 38° C., even better still at room temperature.

Another advantageous form of this resorbable hemostatic adhesive in accordance with the invention is the dry, powdered or sponge form. This form requires no prior preparation before its use.

In order to obtain such a form, the collagen of the invention, obtained in viscous form, is subjected, for example, to lyophilization.

The collagen in liquid form of the invention is evenly distributed into containers (lyophilization trays), under aseptic conditions, leading to lyophilizates with a thickness of 1 to 20 mm, preferably of 2 to 10 mm, and even better still of 3 to 5 mm. The lyophilizate obtained is a cream color and has a slightly elastic, film-forming aspect. It has a water content of 1% to 25%, and preferably of 5% to 15%. The lyophilizates can undergo grinding so as to give a powder of chosen small particle size.

For the powder preparations, it is preferable to have a collagen concentration before lyophilization of about 3 to 6%, or more, so as to ensure better spraying.

The powder after grinding can be distributed into sterile polyethylene vials with a double cap, or another container which allows the sterility to be maintained and which facilitates spraying over the hemorrhagic surface.

In the case of sponges, the collagen concentration should be relatively low, preferably from 1 to 2%.

The film-forming sponges can be packaged individually, after lyophilization, in double bags or in blister packs which are closed by heat-sealing. They are, for example, square, triangular or circular in shape and have a side length or a diameter of 1 cm to 30 cm depending on the uses.

The action of the two dry forms mentioned above is similar to that of the liquid form already described. However, the film-forming sponge also exerts a mechanical action which is often vital in the event of hemorrhage and of bleeding in pools, in particular when abdominal.

This adhesive, whatever its form, in particular viscous liquid, pseudosolid, film-forming sponge or powder, can be combined with active principles, in particular pharmaceutical active principles, such as for example clotting factors which are involved in accelerating hemostasis (such as, for example, thrombin, fibrinogen, factor XII, etc.), or alternatively antibiotics, in particular in the case of bone surgery.

When it contains clotting factors, this adhesive can advantageously be combined with stabilizers such as, for example, polyols (for instance glycerol, or a polyalkylene glycol in which the alkylene radical has from 1 to 4 carbon atoms, such as polyethylene glycol or polypropylene glycol, in particular) or mono-, di- or polysaccharides (such as glucose or sucrose).

As other preparations obtained from the viscous liquid form of collagen according to variant 1 of the method of the invention, mention may be made of:

A pseudosolid patch, as a vehicle for active principles for extra-epidermal local use, or alternatively a cicatrizing spray which is suitable for in particular patients with third-degree burns, as well as a moisturizing mask for cosmetic use.

The patch can incorporate one or more antibiotics, anti-inflammatory agents, antiseptics, antibacterial agents, antifungal agents and antimycotic agents, and other physiologically and pharmaceutically active substances, in particular salicyclic acid or nicotine. These active principles are added at a temperature higher than 40° C. until homogenization is obtained. The mixture is degassed and propelled by a system under pressure, for example, such as a metering pump, and is distributed into sealed packaging (blisters or polyethylene films or the like, for example). After cooling the liquid to room temperature, the packaging is closed by heat-sealing.

The collagen concentrations used in the patch range from 1 to 8%, and preferably from 2 to 5%.

When they are present, the antibiotics have the following concentrations in mg of antibiotic/mg of collagen.

Gentamicin: from 0.01 to 2 mg/mg, even better still from 0.1 to 0.7 mg/mg and preferably from 0.15 mg/mg to 0.5 mg/mg.

Vancomycin: from 0.01 to 2 mg/mg, even better still from 0.1 to 0.7 mg/mg and preferably from 0.15 mg/mg to 0.5 mg/mg.

Pefloxacin: from 0.01 to 2 mg/mg, even better still from 0.05 to 0.5 mg/mg and preferably from 0.075 mg/mg to 0.25 mg/mg.

The thicknesses of the patch can range from. 1 to 5 mm or thicker. Patches 1.5 to 2.5 mm thick are, however, preferred.

The length and width are adjusted depending on the use envisaged.

A patch obtained in accordance with the present invention has a pseudosolid form, is flexible, strong and highly hydrated (water content greater than 80%), and has a translucent appearance.

It is easily applied to internal or external tissue surfaces, whether they are damaged or healthy. In the case of external epidermal uses, the patch can be fixed onto an impermeable hypoallergenic adhesive which allows it to adhere to and be kept on the tissue surface.

The mask is obtained by pouring the collagen, on its own or combined with active principles such as, for example, vitamins (C, E, etc.), a surfactant such as benzalkonium chloride or moisturizers such as glycerol, onto a slow-moving conveyor belt for example, which makes it possible to obtain a homogeneous film of even thickness. After cooling, it is cut up into suitable dimensions in pseudosolid form, and is packaged in hermetic bags (made of aluminum with internal polyethylene linings, for example).

The collagen concentrations which are useful in the preparation of such a mask are in general from 0.2 to 3%, even better still from 0.5 to 2% and preferably from 1 to 1.5%.

The shapes and dimensions are in no way limited. A face mask conventionally has the shape and dimensions of a sheet of A4 type (21×29.7 cm). The thicknesses are generally from 0.5 to 3 mm, and preferably from 1 to 1.5 mm.

The mask has a pseudosolid form, is very flexible, soft to the touch and translucent, and is highly hydrated (water content greater than 97%). It is very easily applied to the skin. One or more dyes and other active principles which are compatible with collagen may be added thereto.

The spray contains the collagen and the active principles in the same concentrations and the same combinations as those described for the patch. It is advantageously packaged in single dose vials or sprayers of 5 to 25 ml which are preferably made of glass or polyethylene.

Before use, the collagen, which is in polymerized form at room temperature, is liquefied by heating in a water bath or in an incubator for 2 to 3 min. in its original packaging. The collagen is sprayed onto the tissue surface from a distance immediately before it sets.

The collagen thus sprayed adheres to the tissue surface and polymerizes very rapidly on this surface, thus forming a hemostatic and cicatrizing homogeneous film. Such a spray is particularly suitable when the aim is a cicatrizing action and/or a sustained release of active substances such as antibiotics, antiseptics, antimycotic agents, anti-inflammatory agents, etc).

This device for spraying from a distance has the advantage of avoiding any contact by the operator with the damaged tissue surface, and thus any risk of possible local infection.

One of the uses of this spray, which is particularly advantageous and is made possible because of the specific properties of the collagen in accordance with the present invention, concerns in particular patients with third-degree burns for whom a guarantee of sterility and of rapidity of care is a necessity.

Like other preparations obtained, from the viscous liquid form of collagen, according to variant 1 of the method of the invention, mention may also be made of: the films or membranes obtained by pouring onto a polyethylene belt, for example, followed by drying under air, in a ventilated incubator or under vacuum. The operating temperature is from 10° C. to 35° C., and preferably from 20° C. to 25° C.

In order to obtain, for example, films with a thickness of less than 1 mm, preferably of 25 μm to 250 μm, collagen solutions are conventionally used in which the concentration ranges from 2 to 5%, preferably from 2.5 to 3.5%. The durations of drying can range from 1 h to 120 h. Advantageously, the durations of drying are about 24 h. Specifically, it is observed that the best results are obtained with relatively slow drying and a relatively low flow rate of air.

The films or membranes thus obtained have a water content of 1 to 15%, and preferably of 5 to 10%. They are cut up under aseptic conditions, either manually or automatically, and packaged in sealed and impermeable packaging.

In a similar manner to the other abovementioned forms, the films or membranes can contain active principles such as antibiotics, antiseptics or also anti-inflammatory agents, etc. They can be produced from acidic (low pH) or neutral collagen.

They are used for external or internal purposes for cicatrizing and/or releasing an active principle, for example a medicinal one. Because of their elasticity upon contact with a hemorrhagic wet surface, they can advantageously form an envelope which adheres to the area of damaged tissue.

In the ophthalmological uses, they are produced from a collagen at pH from 6.8 to 7.2, preferably. In this case, the films or membranes can take the form of a reservoir cylinder or of transparent contact lenses, for a sustained local delivery of active principles to the eyes, for example. When it is desired to use this presentation of collagen in a liquid form (drops/eyewash), the collagen is conserved in capsules or other single dose packaging, which must be reheated before use.

In the case of ophthalmological uses, the collagen concentrations at neutral pH are, for the membranes or lenses, from 1% to 2.5%, and preferably 0.5% to 2%, and, for the eye drops at neutral pH, the collagen concentration is from 0.1% to 1.5%, preferably 0.2% to 0.5%.

A subject of the present invention is thus also the compositions described above for which, by way of illustration only, some of the possible pharmaceutical forms have been cited.

According to a second variant of the method according to the present invention, the extract is subjected, before the mechanical treatment in said mixer with temperature control, to limited proteolysis with a suitable enzyme, with the exception of collagenase, such as for example trypsin, papain, or pepsin, preferably pepsin, as will be set out below. In general, the collagen concentration in the extract is from 0.1% to 2%, even better still from 0.2% to 1%, and most preferably from 0.3% to 0.5%.

Said pepsinized extract (purified, solubilized) is then treated as follows:

Clarifying filtrations are carried out in cascade through membranes with various porosities, for example 100 to 50, and then 20μ, 1μ, up to 0.45 microns.

A new purification step is then carried out by differential precipitations with alkaline earth metal salts, for example with potassium or sodium chloride, preferably with sodium chloride, optionally followed by dialyses against K₂HPO₄ and Na₂HPO₄ phosphate buffers, and finally, by centrifugations in order to recover the precipitated collagen.

The pellet thus obtained is homogenized, by gentle stirring in purified water, the collagen concentration being from 1% to 30%, and then it is washed 2 to 3 times before resuspending in purified water and leaving to stir gently for 48 h at a temperature of 0° C. to 20° C., and preferably a temperature of 0° C. to 10° C.

This suspension is centrifuged or decanted with spin-DRYING. The collagen is collected in a 10⁻²M citric acid solution at low pH of 2 to 5, preferably 2.5 to 3.5, the volume of which is calculated as a function of the chosen final collagen concentration.

The pellet is left to stand in this acid solution without any stirring at low temperature of 0° C. to 10° C. for 2 h to 48 h, and preferably 12 h to 24 h.

The collagen pellet swells, and loses its white coloration so as to become translucent.

The pellet is transferred into a mixer with double transverse cutters, such as, for example, the DITO-SAMA cutter/mixer already mentioned, and then the stirring and shear are gradually increased so as to reach a stirring, preferably of 1000 to 5000 rpm, even better still of 2000 to 3000 rpm, while at the same time controling the increase in temperature so as not to exceed 35° C. preferably 25° C. until total solubilization is obtained. The solution obtained is clear.

Here again, the collagen is prepared from an extract of collagen which is preferably mostly of type I.

According to this variant 2, the solution of native collagen thus prepared and purified can then undergo, advantageously, a sterilization by absolute filtration through a membrane with a 0.22μ porosity at the operating temperature, i.e. according to this variant, preferably at a temperature of 20 to 25° C., or alternatively by adding peracetic acid under conditions which are identical to those already described above, or alternatively by ionizing radiation (2.5 Mrads) on a predried form which is optionally intended to be solubilized in sterile liquid medium in order to be combined with a sterile preparation of active principles which are sensitive to ionizing radiation and which are chosen from the therapeutic classes referred to in the present document.

The optional neutralization is carried out, preferably, after the sterilization step at room temperature. At room temperature, it is observed that a precipitate begins to form from the second hour of neutralization onward. Thus, it is particularly advantageous to conserve the collagen at a temperature of 0° C. to 8° C., and to prepare the combinations or mixtures of the collagen with the other active principles, which are incompatible at low pH (from 1 to 6.5), during the first two hours after neutralization, preferably within the first hour, and even better still within 30 min., and then to begin carrying out in particular the optional operations of freezing, lyophilization or drying under air or under vacuum.

Characteristics of the collagen obtained according to variant 2:

The atelopeptide collagen thus prepared is sterile and in native form. It is in the form of a clear solution which has a low viscosity of 2 cps to 40 cps, and which is at acid or neutral pH if required.

The other characteristics of the collagen thus produced are as follows:

collagen containing 90%, or more, type I collagen, i.e. an α₂(I)₁/α₁(I)₂ ratio of 0.48 to 0.52

denaturation peak: 38° C. to 45° C., as measured by differential thermography (DSC)

total nitrogen: 17.0% to 18.7%

hydroxyproline: from 12% to 13.9%

free of tryptophan, aminoglycans and polypeptides of molecular weight<95,000 daltons

lipids<1%

sulfuric ash<2%

Thus, a subject of the present invention is also such an atelopeptide sterile native collagen.

Preparations based on collagen obtained according to variant 2, and uses. By way of examples, mention may be made of:

Use without modification in liquid form at acid pH, as a tissue moisturizer or combined, in preparations, with cosmetic active principles for example, at concentrations of 0.1% to 3%; in combination in cicatrizing therapeutic preparations, for example in the presence of active principles in dermatology, such as anti-inflammatory agents, local antiseptics, antibacterial agents, antifungal agents, salicylic acid, etc. These formulations are in the form of cream, gel, foam or ointment for external use, the collagen being incorporated therein at concentrations of 1% to 3%. They can also be in the form of a liquid or of a spray for external use, the collagen concentration then being from 0.1% to 1%. The active principles mentioned are used at the known therapeutic doses.

Use at neutral pH in paste or gel form in combination with phosphates and alkaline salts (sodium chloride, for example), the collagen concentration then being from 2% to 7%, for example, in the correction of skin depressions such as wrinkles or fine lines, or alternatively for reinforcing smooth muscles, such as in the case of urinary incontinence, or finally for manufacturing tissue substitutes such as heart valves, for example, or bone substitutes, the collagen then being combined in particular with hydroxyapatite or with coral.

Use at neutral or acid pH in the preparation of a foam which contains a low collagen concentration, and which is particularly useful in surgery and microsurgery for repairing damaged tissue and for ensuring that inter-tissue and parenchymal space is maintained and avoiding anatomopathological adhesions between damaged or lesioned tissues (preferably in parenchymal, cardiovascular, thoracic, plastic or ENT surgery). This foam can incorporate active principles, in particular antibiotics, antiseptics and anti-inflammatory agents, in the same concentrations as those mentioned above for the preparations according to variant 1. Such a foam will be described later in greater detail.

Using this sterile collagen according to variant 2, it is also possible to prepare, in particular with the aid of drying by lyophilization, resorbable hemostatic sponges which are particularly advantageous due to their hemostatic action and for locally vehicling one or more active principles such as in particular anti-infectious agents and/or clotting factors.

Such sponges obtained from collagen at acid pH according to variant 2 are very advantageous, since they are instantaneously soluble once they have been brought into contact with a liquid medium, which allows the immediate release of one or more active principles. The same sponges obtained from a collagen at neutral pH according to variant 2, the appearance of which is microfibrillar, allow sustained release and also exert an advantageous mechanical action on the hemorrhagic surface to which they adhere.

A collagen at acid or neutral pH, at a concentration of 0.1% to 3%, preferably 0.2% to 2% and even better still from 0.5% to 1%, can be used in the preparation of such sponges.

When they are present, the following antibiotics: gentamicin, pefloxacin and vancomycin, are used at the concentrations already mentioned for the preparations of variant 1.

Other active principles can also be present, such as thrombin at a concentration, in International Units per mg of collagen, of 0.1 IU/mg to 20 IU/mg, preferably of 0.5 IU/mg to 10 IU/mg and even better still of 1 IU/mg to 5 IU/mg, or fibrinogen (in mg of fibrinogen per mg of collagen) in a proportion of 0.1 mg/mg to 20 mg/mg, preferably of 0.1 mg/mg to 10 mg/mg and even better still of 0.5 to 5 mg/mg. Other active agents can also be combined therewith, such as other blood derivatives, in particular prothrombin, factor Xa, factor IXa, factor XIII and any other activator or proactivator of intrinsic and extrinsic mechanisms of clotting, optionally combined with other physiologically and pharmacologically active substances such as activators and inhibitors of plasminogen, or antifibrinolytic agents, for example. Other substances can also be combined therewith, for example specific enzymes which regulate collagen resorption, or alternatively crosslinking agents.

The mixtures of collagen according to the invention and of the abovementioned active principle(s) are prepared in containers which are made of stainless steel, glass or polyethylene, and at temperatures of 5° C. to 35° C., preferably of 10° C. to 30° C., even better still of 15° C. to 20° C.

As already mentioned, when it is at neutral pH, the collagen should be used within the 2 hours following its neutralization. The mixture is then distributed into the lyophilization containers which allow layers which are from 1 to 20 mm, preferably from 3 to 15 mm and even better still from 5 to 8 mm thick to be obtained. These preparations can thus be lyophilized as a monolayer according to the following operating parameters: freezing temperature of −5° C. to −60° C., resorption temperature of 20° C. to 50° C., in general of 25° C. to 40° C. and preferably of 30° C. to 35° C.

The preparations of sponges thus lyophilized are white in color, and have a homogeneous, woven, downy appearance. They have a water content of 1% to 25%.

The sponges in accordance with the invention can be used without modification, or can undergo lamination in situ (in the chamber of the lyophilizer) by exerting an individual pressure thereon or, on the outside under aseptic conditions, by passing through a rolling mill. This operation makes it possible to increase the strength of this type of sponge.

Said preparations can be advantageously lyophilized in one or more layers. In this case, the adhesion between the layers preferably occurs during the freezing phase at chosen temperatures of −11° C. to −20° C., preferably of −11° C. to −13° C. A laminate is then obtained in which the two lower layers, for example, can contain collagen at acid pH combined for example with thrombin and with fibrinogen (allowing the release of these products and the formation of a biological adhesive), and in which the upper layer consists, for example, of collagen at neutral pH (making it possible to exert the mechanical action on the hemorrhagic surface as already mentioned). The abovementioned form makes it possible, very advantageously, to vehicle a biological adhesive without any prior preparation, unlike known adhesives. Advantageously, the layer at neutral pH contains antibiotics, this permitting their sustained release, which is particularly advantageous in the case of bone infections. The choice of the order and of the number of layers is immaterial; it is within the scope of persons skilled in the art, who will adjust the latter depending on the uses envisaged.

In these combinations in accordance with the present invention, stabilizers can also be included, such as polyols, polyalkylene glycols or polysaccharides, or alternatively amino acids or other active agents which are compatible with collagen, such as anti-inflammatory agents, for example, or alternatively crosslinking agents as already mentioned, in order to increase, again advantageously, their resorption time. It is also possible to combine therewith other resorbable agents which have a mechanical action and which make it possible to perform sutures, such as cellulose or oxidized cellulose tissues or vicryl® (sold by the company Ethnor).

Moreover, the collagen obtained still according to variant 2 of the method of the invention, at acid or neutral pH, possibly combined with other active principles, can produce a hemostatic powder after lyophilization or drying. The collagen used then has a concentration of 1% to 3%.

A subject of the present invention is thus also the compositions which are described above, and for which some of the possible pharmaceutical presentations, which represent preparations of nonpolymerized collagen obtained according to variant 2, have been illustrated.

Hereafter, nonlimiting examples will be given of some subjects of the present invention, and with reference to the drawings in which:

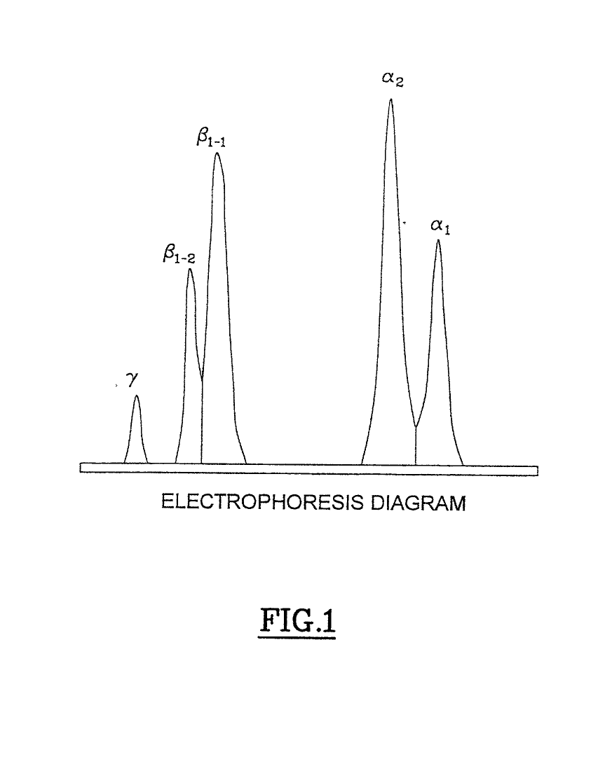

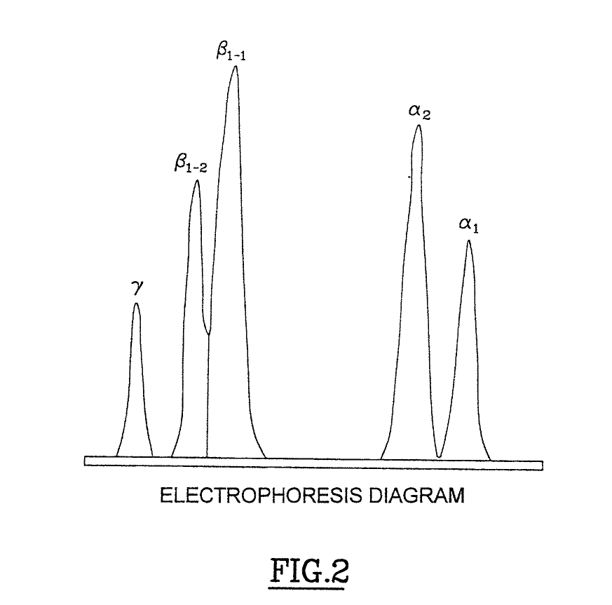

FIGS. 1 and 2 are diagrams of polyacrylamide gel electrophoresis showing the separation of the polypeptide chains of type I collagen, respectively before and after the mechanical and heat treatment of the invention (variant 1).

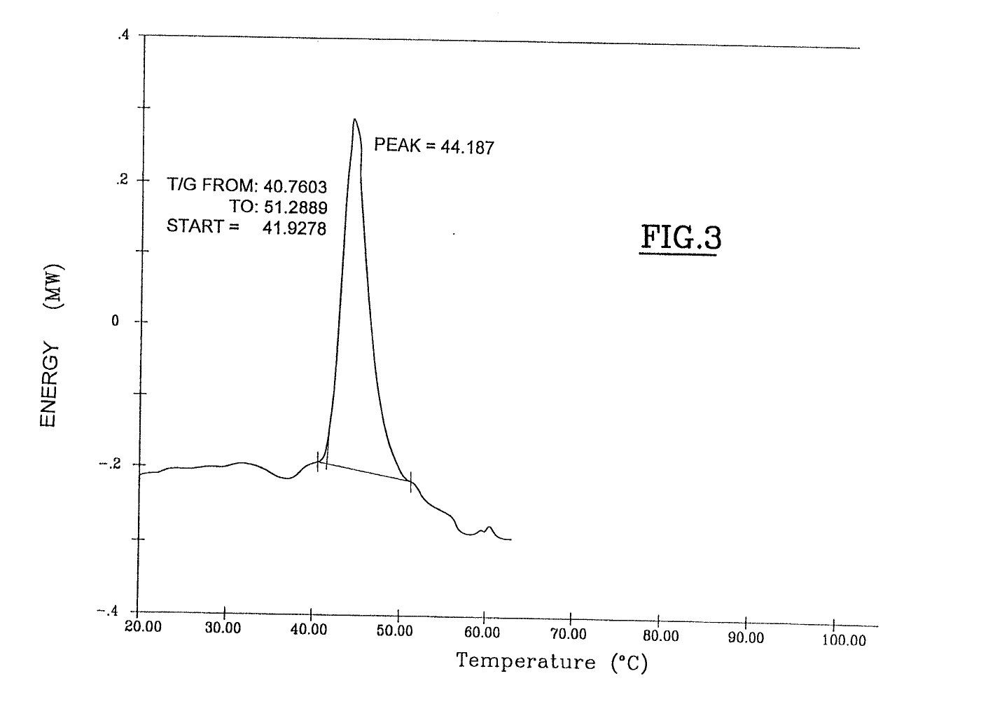

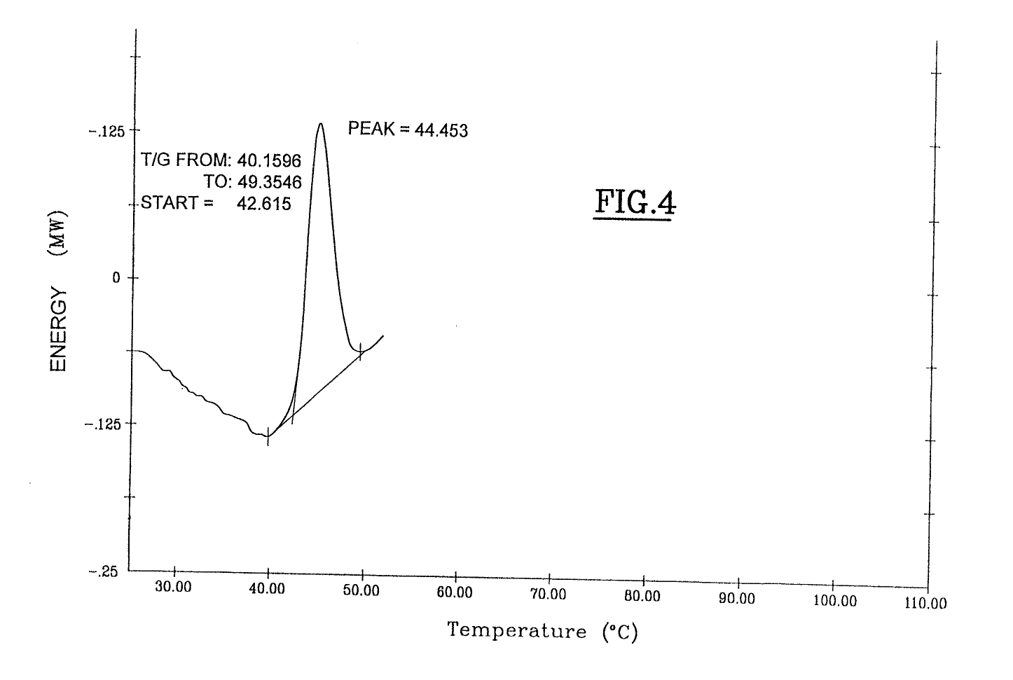

FIGS. 3 and 4 are diagrams of heat denaturation of the collagen demonstrating the native (nondenatured) nature of the collagen, before the mechanical and heat treatment of the invention (variant 1) and after it, respectively.

### EXAMPLES

### Example 1

### Method for Preparing a Collagen with Polymerizing Power (Variant 1: Nonpepsinized Extract)

The extract of collagen at 10%, prepared from ostrich Achilles tendons as above, is transferred into the mixer, with double transverse cutters, already mentioned. Stirring and shear are then carried out at, a speed and rate of 1500 rpm and at room temperature (20° C.) for 7 min. An increase in the viscosity to 15,000 cps is noted. The extract becomes whitish in color and has an elastic appearance. Successive dilutions are then carried out so as to attain a collagen concentration of 6%. After each dilution, the temperature is gradually raised by 4° C. and the stirring speed is gradually raised by 500 rpm, at 3- to 4-min. intervals. When a stirring speed of 3500 rpm and 35 to 36° C. are reached, these conditions are maintained for 3 min. approximately, and then the stirring is stopped and the extract is left to stand at the same temperature of 35 to 36° C. for 30 min. A new dilution (5-fold) is then carried out, and the speed is increased so as to reach 5000 rpm, and the temperature is increased so as to reach 42° C., in 3 to 4 min. The collagen is then pasty and has a viscosity of 17,000 to 20,000 cps. The temperature is increased so as to reach 44° C. Then, these conditions are maintained for 30 sec to 1 min.; the pasty collagen rapidly liquefies, becoming fluid in appearance, its viscosity being reduced to 30 to 50 cps.

The liquefied extract is immediately transferred into a device for filtration under pressure which has been heat-sterilized beforehand, and which is equipped with a double envelope with water circulation allowing the temperature to be maintained at 42 to 45° C. The extract is then thoroughly filtered through two sterile filtration membranes, the first with a 0.45μ porosity and the second with a 0.22μ porosity. The filtrate is collected in a container under pressure equipped with a double envelope, the temperature being maintained at 42 to 45° C. This filtrate is then subjected to an absolute filtration through a membrane with 0.22μ porosity.

The collagen obtained is sterile, as confirmed by the bacteriological tests according to the European Pharmacopeia.

Moreover, the analyses carried out before, during and after the mechanical and heat treatments of the collagen thus obtained show that the amino acid composition of the collagen remains unchanged. Furthermore, no polypeptides of molecular weight lower than 95,000 Da are detected by polyacrylamide gel electrophoresis (FIGS. 1 and 2). It is also noted that the α₂(I)₁/α₁(I)₂ ratio is 0.49, i.e. a collagen containing more than 95% type I collagen.

The thermographic analysis, by differential thermography (DSC) carried out with the aid of the Delta-Series DSC 7 model from the company Perkin-Elmer and performed during the heat and mechanical treatment at 40 to 50° C., confirms the disappearance of the denaturation peak of the collagen, which corresponds to the loss of the trihelical structure of the molecule. However, this disappearance is only temporary, since the reappearance of the denaturation peak is noted after cooling of the collagen extract and its setting (FIGS. 3 and 4). This is the proof that the loss of the trihelical structure of the collagen during the heat and mechanical treatment in accordance with the present invention is reversible in nature.

This set of results proves that the secondary and tertiary structures are maintained, and thus that the collagen thus prepared is in native form.

The other main characteristics and properties of the collagen thus obtained are as follows:

water content: 95%

appearance: viscous liquid at a temperature higher than 38° C. approximately and sticky to the touch; pseudosolid compact and homogeneous gelified mass at a temperature lower than 38° C. approximately.

viscosity: 45 cps in liquid form

total nitrogen: 17.9%

hydroxyproline: 12.8%

free of tryptophan, aminoglycans, and polypeptide chains<95,000 daltons

pH of 5.5

lipids<1%

sulfuric ash<2%

The sterile collagen prepared as has just been described is maintained at 42 to 45° C. for a limited period of time in order to be rapidly distributed without modification into suitable containers or packaging such as vials, syringes, blisters, etc., or after mixing with other active principles, such as clotting factors, which have been sterilized beforehand if required.

When used alone, the collagen thus produced forms a adhesive which has excellent hemostatic properties for uses in general, parenchymal, cardiovascular, thoracic, orthopedic, ENT, urological, hepatic, visceral and gyneco-obstetric surgery, and transplantation and bone marrow surgery; in microsurgery and any other medical field requiring the action of this medicinal form possibly combined with other active principles.

It is kept at room temperature, at which it sets.

This distinguishes it clearly from the other so-called biological adhesives currently available on the market, such as Tissucol® from the company Immuno-AG, Austria, or Biocolle® from the Laboratoires des Fractionnements et des Biotechnologies, France, the dry form of which is necessarily stored at low temperature, about +4° C.

The adhesive based on collagen according to the present invention should be reconstituted in liquid form before being used. To do this, simply heating for 2 to 3 minutes is sufficient, for example in an incubator or in a water bath at 40 to 45° C., followed by slow manual stirring.

Thus, compared with the abovementioned known adhesives, the adhesive based on collagen according to the invention has, besides its advantageous properties, a preparation time which is approximately ten times shorter.

When they are present, the clotting factors are prepared beforehand, before they are added to the collagen, in the following way.

An amount of thrombin or fibrinogen powder corresponding to 2 IU/mg and 1 IU/mg of collagen, respectively, is taken up in a 40 mM solution of calcium chloride (i.e. 1 ml of collagen for 50 IU of thrombin) and in water for injection (WFI). Filtration is then carried out separately through an absolute filtering membrane with a 0.22μ porosity.

The thrombin or the fibrinogen is then added to the sterile collagen which has been neutralized beforehand (pH 7.0), as already mentioned above. The mixture is homogenized and distributed.

The distribution is carried out under pressure, for example using a metering pump, into suitable containers, in particular glass vials or syringes, for example in the case of the preparation of biological adhesive; into heat-sealed blister packs or bags, as is the case, for example, of the patches, films or moisturizing masks, or alternatively into the trays of a lyophilizer in order to obtain a powder or a sponge, etc., as described above.

### Example 2

### Sterile Liquid Biological Adhesive Containing Collagen+Thrombin

Using the product obtained in Example 1 in a sterile liquid form, a composition containing 2 ml of 5% collagen and an amount of thrombin corresponding to 200 IU is prepared in a glass vial.

At the time of its use on a hemorrhagic surface, it sets and adheres to the tissue surface, allowing the closing of the lesion or of the incision, and it acts as a contact hemostatic agent which causes the bleeding to stop.

### Example 3

### Powder Containing Sterile Collagen+Fibrinogen

Using the product obtained in Example 1, and after lyophilization and grinding of the collagen combined with the fibrinogen, in sterile powdered form, 360 mg of collagen containing 700 IU of fibrinogen per vial are distributed into sterile polyethylene vials.

This powder, when applied by spraying to a hemorrhagic surface, particularly in microsurgery in areas where access is difficult, makes it possible to stop the bleeding through its hemostatic action on the intrinsic and extrinsic pathways of clotting, and adheres to the hemorrhagic surface.

### Example 4

### Sponge Containing Sterile Collagen+Thrombin

Starting with 35 ml of sterile liquid collagen, which has a concentration of 1%, and which is as obtained in Example 1 and then neutralized, it is distributed into trays or blister packs of 5×7 cm. A lyophilization is then carried out, and then the trays or blister packs are closed under aseptic conditions, these trays or blister packs then being packaged in sterile double bags.

The sponge thus produced makes it possible to cover an area of 35 cm², and corresponds to an amount of collagen of 350 mg incorporating 700 IU of thrombin.

This sponge has a rapid hemostatic action which combines the action of the collagen with that of the thrombin on the mechanisms of clotting, and adheres to the hemorrhagic surface allowing a considerable mechanical action to be exerted. Its film-forming aspect makes it possible to cover large hemorrhagic areas.

### Example 5

### Patch Containing Collagen (Pseudosolid)+Metronidazole

The anti-infectious agent metronidazole, in liquid form and sterilely prefiltered, is added to the 2% collagen obtained in Example 1 in liquid form at 42 to 45° C. and neutralized as mentioned above, in a proportion of 0.05 mg/mg of dry collagen.

After cooling, the collagen which is in the form of 1 mm-thick clear and homogeneous pseudosolid gel is subjected to sterilization by irradiation with beta rays at 2.5 Mrad, Laboratoires Caric Orsay, France.

It is noted, surprisingly, that the gel prepared from the collagen in accordance with the present invention “does not break”, i.e. does not separate into two phases, unlike a conventional gel undergoing the same treatment of sterilization by irradiation.

This patch, when applied to an infected wound, has a topical and protective action, acting as a disinfectant due to the presence and to the release of metronidazole.

### Example 6

### Atelopeptide Collagen with Polymerizing Power (Variant 1)

Using a 10% extract of purified collagen originating from fresh ostrich Achilles tendon, the following procedure is carried out:

the collagen is diluted by adding a solution of 10⁻²M citric acid at pH 2.5 until a collagen concentration of 1% is obtained,

the pepsin is added; 1/10 weight/weight of dry collagen,

it is left to act at room temperature (20° C.) for 7 hours,

on the 7th day, the collagen is neutralized with sodium hydroxide (30%) in order to obtain a pH of 7.2. The collagen precipitates, the noncollagen proteins and the pepsin residues which remain in solution being removed by centrifugation with a spin-dryer/decanter device such as, for example, the one sold by the company Rousselet under the reference RC40,

the pellet is washed in demineralized water, followed by spin-drying, the weight of water corresponding to 10 times the weight of the pellet, and then a collagen pellet is obtained in the form of humid fibers,

this operation is repeated three times under the same conditions, fibers of collagen with a water content of 70% thus being obtained,

the pellet is solubilized in 10⁻²M citric acid at pH 2.5,

the mixture is homogenized until total solubilization occurs, and an extract of collagen with a concentration of 10%, which is clear and has a viscosity of 6000 cps, is obtained,

the extract is transferred into the mixer with double transverse cutters already mentioned, and the procedure is continued according to the teaching of Example 1, until an approximately 5% collagen is obtained.

This sterile atelopeptide native collagen with polymerizing power is particularly advantageous in ophthalmological uses, and can be in the form of a lens, of flexible membranes or of drops/eyewash, as already described above. Moreover, the absence of potentially immunogenic telopeptides makes it especially attractive.

### Example 7

### Collagen Foam (Variant 2)

The foam is prepared from a pellet of wet fibers of collagen, with a water content of 70%, which is treated according to variant 2 already described. This pellet is homogenized and is maintained for 48 h with gentle stirring at +4° C. in purified water. After centrifugation, the pellet is taken up in a solution of 10⁻²M citric acid (pH 2.5), the collagen concentration being 2.5%. After standing for 24 h at 4° C., the pellet is transferred into the mixer with double transverse cutters. The stirring speed and shear rate and the temperature are gradually increased, and 2000 rpm and a temperature of 25° C. are reached. The extract is then filtered through an absolute membrane with a 0.22μ porosity at the operating temperature of 25° C.

Benzalkonium chloride is added, at a concentration of 1.5 g/l, to the sterile collagen thus obtained at 2.5%. The mixture is subjected to stirring progressing from 500 rpm to 2500 rpm at room temperature, the extract thickens and becomes white in color, and the foam in accordance with the present invention is thus obtained.

The foam is distributed in metering pumps of 10 ml or more optionally equipped with a catheter which allows its administration in areas where access is difficult, as is often the case in microsurgery.

Thus conserved, this foam is stable when stored at room temperature for at least 24 months. In the event of the presence of a heat sensitive active principle, it is preferable to maintain the storage temperature for the foam thus produced at 4 to 10° C.

This foam is advantageously used in particular in parenchymal and thoracic surgery. It is particularly useful in maintaining the inter-tissue and parenchymal spaces, and makes it possible to avoid anatomo-pathological adhesions between the damaged tissues.

### Example 8

### Stratified or Monolayer Hemostatic Sponges (Variant 2)

The sponges are produced from collagen obtained according to variant 2, as in Example 7, which is sterilized by absolute filtration through a membrane with a 0.22μ porosity.

In order to obtain a monolayer sponge which also comprises a clotting factor, for example, the following procedure is carried out:

35 ml of liquid collagen, at a concentration of 1%, which has been mixed beforehand with sterile thrombin (700 IU) or with sterile fibrinogen (350 IU) and then neutralized, are distributed into blisters of 5×7 cm.

each of the abovementioned collagen combinations (the freezing and resorption temperatures being −25° C. and 35° C., respectively) are conventionally and separately lyophilized.

The sponge containing collagen at neutral pH thus obtained has a downy, slightly fibrous appearance, and is respectively white and cream in color in the presence of fibrinogen and thrombin.

This sponge has a hemostatic action which is linked to the extrinsic and intrinsic action of the collagen on the mechanisms of clotting, which is further reinforced by the hemostatic action of the clotting factors, namely thrombin and fibrinogen.

Moreover, the collagen at neutral pH as obtained according to variant 2, since it is insoluble, exerts a mechanical action on the hemorrhagic tissue surface which the clotting factors cannot exert when they are used alone, in lyophilized form.

In addition, and as already mentioned above, in such a presentation in accordance with the present invention, the activity of the clotting factors is totally preserved, unlike conventional preparations (such as, for example, Tachocomb®, Hafslund Nycomed, described in document EP-A-59 265), in which the latter are sterilized by ionizing radiation and are thus partially denatured.

In order to obtain a stratified sponge, the following procedure is carried out:

18 ml of liquid collagen at pH 5, which is at a concentration of 0.5% and which has been mixed beforehand with thrombin (700 IU) and with 1 ml of 40 mM calcium chloride, are distributed per blister of 5×7 cm, and then

gentle freezing is carried out at −10° C., and then 18 ml of 0.5% liquid collagen at pH 5, which has been mixed beforehand with fibrinogen (350 IU), are added to this first layer, and then

gentle freezing is again carried out under the same conditions, and a final layer of 1% liquid collagen, which this time is at neutral pH, is added to the second layer thus obtained, and finally

lyophilization is carried out, as described above in the case of the monolayer sponge.

Thus, a three-layered stratified sponge is obtained which has the following notable properties:

once applied, the collagen at acid pH is immediately solubilized, thus releasing the clotting factors which form a hemostatic adhesive on the hemorrhagic surface,

the external layer at neutral pH also exerts a mechanical action, thus reinforcing the action of the hemostatic adhesive, all the more so if the hemorrhagic area is large.

Moreover, this sponge in accordance with the invention has the advantage of requiring no preparation prior to its use.

This type of hemostatic presentation is particularly suitable in general, parenchymal, cardiovascular, thoracic, plastic, orthopedic, ENT, urological, hepatic, visceral and gyneco-obstetric surgery, transplantation and bone marrow surgery, and microsurgery, and generally in any field requiring such a hemostatic action.

### Example 9

### Sponge+Vancomycin (Variant 2)

The following procedure is carried out:

6 l of a 1.4% solution of collagen as obtained according to variant 2 of the method in accordance with the present invention are introduced into a sterile container, and neutralized by adding sodium hydroxide, then

1 I of a solution of vancomycin hydrochloride at 42 g/l is added to the solution of collagen which is maintained at room temperature, within the hour following its neutralization, then

the solution is homogenized, and then, with the aid of a metering pump, the resulting mixture is transferred, through a filtering membrane with a 0.22μ porosity, into lyophilization containers in a proportion of 70 ml per 10 cm-sided container, and next

the cycle of freezing down to −25° C. is begun, followed by the cycle of sublimation and of desorption up to +35° C.,

sterile sponges which are 7 mm thick and which contain collagen in a proportion of 8.4 mg of collagen/cm² and 4.2 mg of vancomycin hydrochloride/cm², or sponges which are 1 to 2 mm thick are finally obtained, by exerting an automatic pressure on the surface of the layers inside the lyophilizer in order to obtain a laminated sponge, and next

the blisters are heat-sealed and packaged in sterile polyethylene double bags.

Identical sponges in accordance with the invention are obtained incorporating gentamicin, pefloxacin, or other antibiotics or anti-infectious agents in place of the vancomycin.

These sponges, which locally and gradually release their active principle(s), are particularly useful in the treatment of many pathologies, in particular bone, visceral, traumatological and surgical pathologies, in particular in the prevention or treatment of possible microbial contaminations.

These sponges containing collagen in accordance with the present invention, besides their excellent tolerance and biodegradability, have the great advantage of incorporating vancomycin which has been sterilized by filtration, as described above, and which has thus conserved all its activity, unlike conventional preparations, in which this antibiotic is partially denatured by sterilization with ionizing radiation.

1. A method for preparing a collagen from a solubilized and purified, optionally pepsinized, extract of nonsterile collagen, characterized in that it comprises the steps consisting:
(i) in stirring and shearing said extract in a mixer with double transverse cutters while increasing in stages the stirring speed, without exceeding 10,000 rpm, and the temperature from 2° C. to 10° C., preferably 3° C. to 5° C., so as to increase the initial ambient temperature up to a controlled maximum temperature, preferably lower than 50° C., and then
(ii) in sterilizing in liquid medium said extract, as a result of which a collagen which is sterile and in native form is obtained.

2. The method as claimed in claim 1, the solubilized and purified extract not being pepsinized, characterized in that, in step (i), a dilution of the extract is carried out at each of said stages at which the viscosity of said extract reaches a chosen high value of 15,000 to 20,000 cps, and, when said maximum temperature is 42 to 44° C., said extract is left to stand just before step (ii).

3. The method as claimed in either of claims 1 and 2, characterized in that, before step (ii), said extract is thoroughly filtered at said controled maximum temperature through membranes with a porosity of 0.45μ to 0.22μ.

4. The method as claimed in claim 1, said solubilized and purified extract being pepsinized, characterized in that, in step (i), the maximum temperature reached being 35° C., preferably 25° C., and the stirring speed being from 1000 to 5000 rpm, preferably 2500 rpm,
clarifying filtrations are carried out in cascade through membranes of various chosen porosities, up to 0.45μ, and then
differential precipitation of the filtrate is carried out with sodium chloride.

5. The method as claimed in either of claims 3 and 4, characterized in that, in step (ii), an absolute filtration is carried out through a membrane with a porosity of 0.22μ, or the extract is sterilized by adding peracetic acid.

6. The method as claimed in any of one of the preceding claims, characterized in that it comprises a step of neutralization of said extract before or after step (ii).

7. The method as claimed in any one of the preceding claims, characterized in that the collagen is of fibrillar type, preferably a collagen mostly of type I.

8. The method as claimed in any one of the preceding claims, characterized in that the extract of collagen is obtained from dermides, from connective tissues or from animal tendons, in particular rabbit dermides and ostrich Achilles tendons.

9. A native type I collagen, which can be obtained by a method as claimed in any one of claims 5 to 8, characterized in that it has the following characteristics or properties:
α₂(I)₁ /α₁(I)₂ ratio of 0.48 to 0.52
sterile according to the standard of the European Pharmacopeia
total nitrogen: 17.0% to 18.7%
hydroxyproline: from 12% to 13.9%
free of tryptophan, aminoglycans and polypeptides of molecular weight<95,000 daltons
lipids<1%
sulfuric ash<2%

10. A pharmaceutical and/or parapharmaceutical and/or medico-surgical and/or ophthalmological and/or cosmetic composition comprising a collagen as claimed in claim 9 on its own or combined with at least one other active principle.

11. The composition as claimed in the preceding claim, characterized in that said other active principle is chosen from the group comprising clotting factors, for example thrombin, anti-infectious agents, in particular metronidazole, antibiotics, in particular macrolides such as gentamicin, glycopeptides such as vancomycin, and fluoroquinolones such as pefloxacin, acylcarboxylic or oxicam derivative steroidal and nonsteroidal anti-inflammatory agents, growth factors, in particular bone growth factors such as somatotropin, and epidermal growth factors such as EGF (epidermal growth factor), surfactants, moisturizers, stabilizers and vitamins.

12. A pharmaceutical presentation of a composition as claimed in either of claims 10 and 11, characterized in that it is chosen from the group comprising gels, in particular injectable gels and pseudosolid gels, foams, eyewashes, monolayer or stratified sponges, powders, plates or bands, masks, sprays, films, membranes, sheets and threads, in particular for sutures.

13. The use of a collagen as claimed in claim 9 in the manufacture of a composition intended for pharmeutical and/or parapharmaceutical and/or medico-surgical and/or ophthalmological and/or cosmetic treatment of a human or animal body.
